(19) United States
(12) Patent Application Publication
(10) Pub. No.: US 2011/0046474 A1
(43) Pub. Date: Feb. 24, 2011

(54) METHOD TO CORRECT INTERFERENCE SIGNALS IN CARDIAC NOISE-TRIGGERING OF MEDICAL DATA ACQUISITION, AND MEDICAL SYSTEM OPERATING ACCORDING TO THE METHOD

(76) Inventor: Juergen Roessler, Erlangen (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/859,469

(22) Filed: Aug. 19, 2010

(30) Foreign Application Priority Data
Aug. 19, 2009 (DE) 10 2009 038 059.0

(51) Int. Cl. A61B 5/055

(52) U.S. Cl. 600/413

(57) ABSTRACT

In a method and medical data acquisition system for the correction of interference signals (in particular those caused by gradient coils) in a magnetic resonance apparatus given the determination of a point in time in the cardiac cycle of a patient by the acquisition of cardiac noises with a sound sensor arranged on the patient, a calculated and/or pre-measured interference signal that describes the interference noise of the gradient coils is obtained and is subtracted from a raw signal measured by the sound sensor.

### BACKGROUND OF THE INVENTION

1. Field of the Invention

The present invention concerns a method to correct interference signals (in particular those caused by gradient coils) in a magnetic resonance apparatus given the determination of a point in time (in particular the R-spike) in the cardiac cycle of a patient, and concerns an associated magnetic resonance apparatus.

2. Description of the Prior Art

In medical imaging it is known—in particular in acquisitions in the heart region—to trigger the data acquisitions at a specific point in time in the cardiac cycle for a better comparison capability among individual images. In magnetic resonance it is also frequently necessary to conduct such a heart triggering (“cardiac gating”) when series of images to be compared are acquired. For this purpose, an electrocardiogram is usually obtained, and then a corresponding trigger device is triggered at the R-spike (which is easily detectable). The acquisition of electrocardiograms within the scope of magnetic resonance, however has a number of disadvantages since, in addition to the temporally variable magnetic fields (in particular the gradient fields) that occur in a magnetic resonance device, additional effects are present that make the evaluation of a conventionally acquired electrocardiogram difficult. For example, a movement of the electrodes in the basic magnetic field occurs due to the breathing and—since blood ultimately represents a moving conductor—the T-wave is often not measured accurately due to the ejection volume of the heart.

With regard to other image acquisition modalities, in particular radiological imaging and x-ray imaging, in U.S. Pat. No. 4,546,777 it was proposed to measure (detect) cardiac noises by means of a sound sensor arranged on the patient, wherein two different cardiac noises occur within one cardiac cycle. A method is disclosed in the aforementioned patent there with which it is possible to differentiate these two cardiac noises from one another and to derive trigger signals for the aforementioned radiological imaging based on the signals acquired with the sound sensor.

Previously, the use of a cardiac sound sensor in the field of magnetic resonance has not been possible without further measures, since very loud noises occur during data acquisition in a magnetic resonance apparatus, such that a sound measurement at the heart is enormously disrupted by this noise. Thus, detection of the R-spike—whose relationship to the two measured cardiac noises is known—is either drastically distorted due to the noise interference or can not be done at all. The gradient coils are primarily responsible for the high noise volume and the varying background noise.

### SUMMARY OF THE INVENTION

An object of the present invention is to provide a method with which the disruptive influences on a sound measurement in the acquisition of cardiac noises in a magnetic resonance system can be corrected so that a point in time of the cardiac cycle—in particular a trigger signal—can be reliably derived from the cardiac noises.

This object is achieved according to the invention by a method for the correction of interference signals (in particular those caused by gradient coils) in a magnetic resonance apparatus for the determination of a point in time—in particular the R-spike—in the cardiac cycle of a patient by the acquisition of cardiac noises by means of a sound sensor arranged on the patient, wherein a calculated and/or pre-measured interference signal that describes the interference noise of the gradient coils is subtracted from a raw signal measured by the sound sensor.

A method is accordingly achieved with which the application of a sound sensor for cardiac noises is enabled even within the scope of magnetic resonance imaging, by a calculated and/or pre-measured interference signal being subtracted from the raw signal measured by the sound sensor so that a corrected heart signal is produced that can be further evaluated to determine the point in time in the cardiac cycle (for example as is known from U.S. Pat. No. 4,546,777). The interference signal thus in particular indicates the variable portion of the background noise in a magnetic resonance apparatus, or at least a good approximation thereof, wherein in particular the gradient coils are responsible for the variable background noise. However, it is naturally also possible for the interference signal to also include additional occurring noise effects that are not only triggered by the effect of the pulse sequence required for magnetic resonance measurement, but can also be ascribed to other sources.

The invention consequently enables the detection of a point in time in an electrocardiogram or in a cardiac cycle—for example the detection of the R-spike—by means of sound sensors even during a magnetic resonance measurement, in spite of noise interference. A better and more precise triggering of the image acquisition is thus possible; a better image quality in exposures of the heart is also achieved as a result.

As mentioned, the gradient coils are a primary cause for disruptive noises interfering with the measurement in a magnetic resonance apparatus. Therefore the interference signal can advantageously describe the interference noises of the gradient coils, wherein in particular the pulse responses—thus the created interference in the heart sound sensor—can be estimated, measured or adaptively determined based on the gradient activation, such that during the magnetic resonance imaging the interference in the raw heart signal can be estimated using the operating parameters of the gradient coils and be subtracted from the raw signal. The noise interference in the raw heart signal is thereby markedly reduced and a detection of the point in time—in particular of the R-spike—is significantly facilitated.

The interference signal can be computationally determined, in particular with linear dependence on the applied gradient currents. The applied gradient currents are consequently multiplied with coefficients, in particular with a linear dependency. A measured pulse response describing the sound injection characteristic of the gradient coil in the sound sensor is convolved with the current applied at the gradient coil to determine the interference signal for each gradient coil. In this case the coefficient is thus the pulse response that reflects which noise interference is created at the corresponding gradient coil given a unit current. For example, such a coefficient can be measured as explained in further detail in the following.

The interference also signal can be determined based on measurement data acquired within the scope of a calibration measurement. A measurement is thus conducted in advance. This measurement can either directly determine the interference signal or can be used to calculate it. Although simulations or theoretical calculations to determine the interference signal are within the scope of the present invention, a calibration measurement represents an information source about the type of interference signal that is particularly close to reality and simple to obtain, such that a qualitatively high-grade correction is achieved.

For example, before beginning the image acquisition with the magnetic resonance apparatus and given a positioned patient and sound sensor, the gradient coils are individually activated with a predetermined current and/or current curve and the measurement data are acquired during this current feed. The injection of the noise interference is thus sampled for specific operating parameters (here the applied currents) of the gradient coils, such that—through a few measurements—a database is produced that can form the basis for the correction of the raw signal for all following image acquisitions. Measurements are thereby advantageously made with sound sensor already arranged on the patient so that its properties with regard to the sound injection can equally be taken into account. Naturally it is also conceivable to use the at least one pulse sequence in advance, which is used later for MR data acquisition, to operate the magnetic resonance apparatus so that the acquired measurement data can be used (immediately or after a post-processing) as an interference signal to be subtracted.

In calibration measurements of the patient it is generally expected that the heart beat to be measured (detected) later. Therefore, according to the invention it can be advantageous for measurement data to be acquired and averaged repeatedly and under the same operating parameters of the magnetic resonance apparatus that will be used for diagnostic data acquisition, in particular the same currents in the gradient coils. In this way it is ultimately possible to suppress the interfering acoustic signal of the heart in the calibration measurement by it being simply averaged out. A measurement is already possible without any problems with the patient in the position in which the sound signals of the heart should also be measured, such that an excellent correction can be achieved.

It is also possible for measurement data to be acquired within the scope of the calibration measurement using a phantom provided with the sound sensor. In an advantageous embodiment of the method according to the invention, the measurement data of the phantom can be used for an advance correction that is specific to the employed magnetic resonance apparatus while a fine calibration then ensuing from measurements of the actual patient. For a specific magnetic resonance apparatus, it is thus possible to initially acquire base or coarse measurement data using a phantom (for example a simulation of a patient) from which correction values are determined that are in each case subtracted from the raw signal of the sound sensor. The raw signal that is thus corrected in advance is then more finely corrected using the interference signal determined at the actual patient within the scope of the calibration measurement, this interference signal now advantageously already being in a range similar to that of the sound signal of the heart. This interference signal obtained from the fine calibration is accordingly subtracted from the raw signal corrected in advance in order to ultimately obtain a corrected cardiac signal, which can then be evaluated with regard to the at least one point in time in the cardiac cycle and, for example, be used to determine a trigger signal.

As mentioned, at least one pulse response can be determined from the measurement data. The pulse responses determined in this way can then be used in the determination of the interference signal under consideration of the gradient currents, for example. For example, a step response can be measured in order to determine a pulse response therefrom, defined as a response to a Dirac pulse. Other methods are also conceivable.

In addition to the method, the present invention also concerns a magnetic resonance apparatus having a trigger device with a sound sensor to acquire cardiac noises of a patient and a control device that is fashioned to implement the method according to the invention. All embodiments cited with regard to the method according to the invention can analogously be transferred to the magnetic resonance apparatus according to the invention.

In this way a magnetic resonance apparatus is achieved in which—in spite of the high noise interference—an image data acquisition is triggered based on a heart signal that is acquired by a sound sensor arranged on the patient and corrected according to the invention. Interference-prone EKG measurement devices that require additional electronics are no longer necessary.

### BRIEF DESCRIPTION OF THE DRAWINGS

FIG. 1 is a flowchart of the method according to the invention.

FIG. 2 schematically illustrates a magnetic resonance apparatus according to the invention.

### DESCRIPTION OF THE PREFERRED EMBODIMENTS

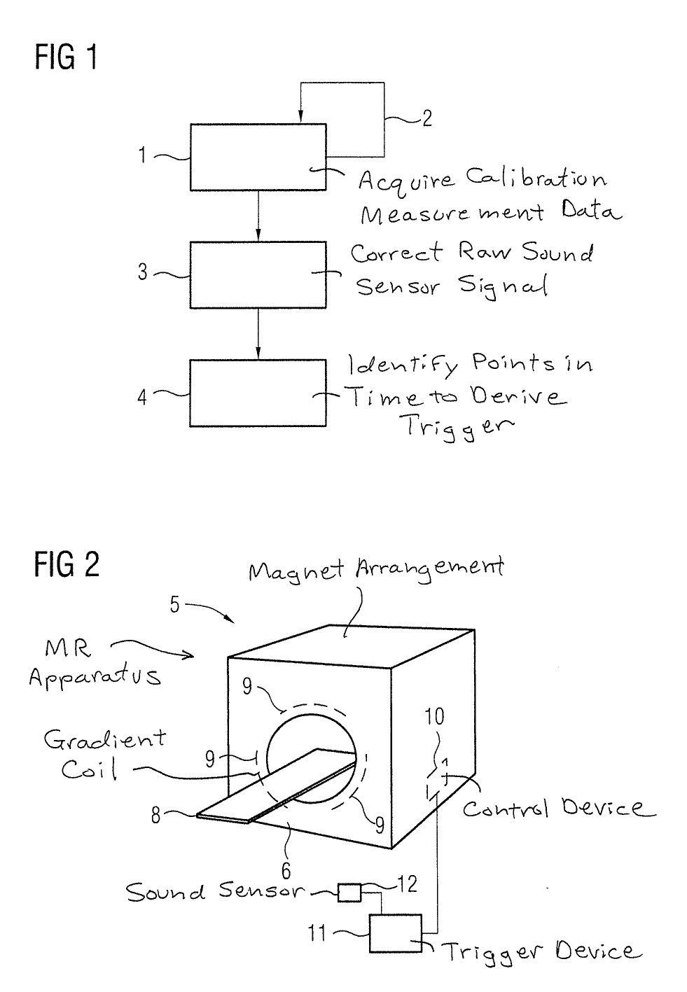

The method according to the invention, explained in detail based on the embodiment shown in the flowchart in FIG. 1, ultimately serves to determine a point in time in a cardiac cycle from which a trigger signal (for example) can be derived.

In spite of the enormous background noise in a magnetic resonance apparatus, a sound sensor is used for this purpose in accordance with the invention, the sound sensor being arranged on a patient in the region of the heart in order to acquire raw signals containing the cardiac noises. Without a correction, a significant noise interference is present that is primarily caused by the gradient coils of the magnetic resonance apparatus. The exemplary embodiment of the method according to the invention that is shown concerns a method that is simple to realize in order to correct the sound injection due to the gradient coils at the actual patient.

For this purpose, according to the invention, measurement data are initially acquired (Step 1) within the scope of a calibration measurement before the diagnostic image data acquisition, after the sound sensor has been arranged in its correct position at the heart of the patient and the patient has been placed in the patient receptacle. For this purpose, the individual gradient coils of the magnetic resonance apparatus are respectively fed with specific currents in a time-offset manner, these currents serving as reference currents (thus in particular they are uniform currents). The signal of the sound sensor is read out for each coil so that three sets of measurement data are obtained that represent a pulse response, thus the generated sound interference in the sound sensor upon application of the current at the respective gradient coil. In order to correct the already-present cardiac signal of the patient that is created by cardiac noise, multiple measurements are conducted (as indicated by Arrow 2) that are then averaged in order to then determine for each gradient coil a pulse response Uᵢ(t) that no longer contains the actual cardiac noise. As is also the case in the following, t symbolizes the time dependency.

It should be noted that, within the scope of the method according to the invention, an advance correction of the raw signal acquired by the sound sensor can also ensue, that is specific to the magnetic resonance apparatus, with regard to obtain a raw signal that is corrected in advance, which is not shown in detail in FIG. 1. For this purpose, the sound sensor is arranged once or during different, longer time periods on a phantom which, for example, can simulate a patient. Measurement data can likewise then be determined that can be used to determine a rough correction or advance correction. Only a fine calibration is then still necessary at the actual patient, such that an extreme difference in orders of magnitude no longer needs to be accounted for if such an embodiment is used.

The results of Step 1 are consequently three pulse responses Uᵢ(t) which, in Step 3 of the method according to the invention, can be used during the diagnostic image data acquisition to correct the raw signal (possibly the raw signal corrected in advance). For this purpose, an interference signal S(t) is initially determined depending on the currents Iᵢ(t) applied to the gradient coils and used for magnetic resonance measurement:

S(t)=Uₓ(t)*Iₓ(t)+U_{y(t)}*I_{y}(t)+U_{z}(t)*I_{z}(t)

The pulse responses Uᵢ(t) as coefficients are thus linearly convolved with the currents Iᵢ(t) applied to the gradient coils in order to determine the interference signal describing the interference noise of the gradient coils. This interference signal is now subtracted from the raw signal (which is possibly corrected in advance) to correct the raw signal R(t) in order to obtain the cardiac signal H(t) that is to be further evaluated:

H(t)=R(t)−S(t)

The corrected raw signal that is acquired in this way (thus the cardiac signal) can then be reliably evaluated in order to determine the points in time of the two cardiac noises as well as points in time of the cardiac cycle derived from these (Step 4). For example, from these points in time trigger signals can be derived in order to always acquire image data from the heart at a fixed, predetermined point in time in the cardiac cycle.

FIG. 2 shows a magnetic resonance (MR) apparatus 5 according to the invention in a basic drawing. A magnet arrangement 6 surrounds a patient receptacle 7 into which a patient on a patient bed 8 can be inserted. In addition to the basic magnet (not shown in detail), the magnet arrangement 6 also includes a radio-frequency coil arrangement (not shown in detail) and gradient coils (indicated at 9) respectively for the x-, y- and z-directions.

The operation of the magnetic resonance apparatus 5 is controlled by a computerized control device 10 to which a trigger device 11 is also connected with which trigger signals are generated to generate image exposures each at a specific point in time in the cardiac cycle. A sound sensor 12 is thereby associated with the trigger device 11, which sound sensor 12 is placed in the heart region on a patient to be examined in order to acquire a raw signal representing his cardiac noise.

The control device 10 is fashioned to implement the method according to the invention as explained in FIG. 1, for example. An interference signal is accordingly determined that is subtracted from the raw signal in order to correct the noise interference of the gradient coil 9 so as to obtain an cardiac signal in a form that can be evaluated so that points in time in the cardiac cycle and trigger signals can be derived.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for correcting cardiac noise signals acquired in a medical data acquisition system, comprising the steps of:
acquiring a raw signal from a sound sensor arranged on the patient that contains a signal component representing cardiac noises in the patient, in an environment in a medical data acquisition system in which the patient is located in which interference noise exists;
prior to acquiring medical data from the patient in said medical data acquisition system, predetermining, in a procedure selected from the group consisting of a calculation procedure and a measurement procedure, an interference signal that describes said interference noise, and subtracting said interference signal from said raw signal to generate a corrected raw signal that is substantially free of said interference noise; and
deriving a trigger signal from the corrected raw signal and triggering acquisition of medical data from the patient in said medical data acquisition system using said trigger signal.

2. A method as claimed in claim 1 comprising evaluating said corrected raw signal to identify a point in time in the cardiac cycle of the patient, and deriving said trigger signal to cause triggering of said acquisition of medical data at said point in time.

3. A method as claimed in claim 2 comprising determining said point in time by analyzing the corrected raw signal to identify an occurrence of the R-spike therein.

4. A method as claimed in claim 1 wherein said medical data acquisition system is a magnetic resonance apparatus comprising gradient coils, and wherein said interference noise is generated by operating said gradient coils with respective gradient currents supplied thereto, and comprising predetermining said interference signal by a calculation with a linear dependence on the respective gradient currents.

5. A method as claimed in claim 4 comprising supplying respective current pulses to said gradient coils and, with said sound sensor, detecting respective pulse responses of said interference noise resulting from the respective operation of said gradient coils with said current pulses, and calculating said interference signal by convolving the respective pulse responses with the respective current pulses.

6. A method as claimed in claim 1 comprising implementing a calibration measurement prior to said medical data acquisition to obtain calibration measurement results, and predetermining said interference signal from said calibration measurement results.

7. A method as claimed in claim 5 comprising, in said calibration measurement, positioning said patient in said data acquisition system, and positioning said sound sensor on said patient, at respective positions corresponding to positions that will be used to acquire said medical data from the patient, and operating said medical data acquisition system in said calibration procedure in a same manner of operation that will be used for acquiring said medical data from the patient.

8. A method as claimed in claim 7 comprising implementing a plurality of calibration measurements in said calibration procedure and averaging said calibration measurements to generate said interference signal.

9. A method as claimed in claim 7 wherein said medical data acquisition system is a magnetic resonance apparatus comprising gradient coils, and wherein said interference noises are generated by operating said gradient coils with respective currents, and comprising implementing said calibration procedure by supplying respective currents to said gradient coils in said calibration procedure that are the same as currents that will be supplied to the gradient coils to acquire said medical data from the patient.

10. A method as claimed in claim 6 comprising placing a phantom in said medical data acquisition system that represents the patient, and placing the sound sensor on said phantom, and acquiring calibration measurement data in said calibration procedure from said phantom.

11. A method as claimed in claim 10 comprising, after said calibration procedure, acquiring further calibration data from the patient in the medical data acquisition system, and limiting the further calibration data acquired from the patient using the measurement data acquired from the phantom.

12. A method as claimed in claim 6 comprising operating said medical data acquisition system in said calibration procedure to obtain a pulse response with said sound sensor to said interference noise, and determining said interference signal from said pulse response.

13. A medical data acquisition system comprising:
a medical data acquisition unit adapted to receive a patient therein, said medical data acquisition unit generating interference noise during operation thereof to acquire medical data;
a sound sensor arranged on the patient in said data acquisition unit, said sensor generating a sensor signal that contains a signal component representing cardiac noises in the patient, contaminated by said interference noise exists;
a triggering signal generator configured to, prior to acquiring medical data from the patient in said medical data acquisition system, predetermine, by calculation or measurement, an interference signal that describes said interference noise, and to subtract said interference signal from said raw signal to generate a corrected raw signal that is substantially free of said interference noise; and
said trigger signal generator being configured to derive a trigger signal from the corrected raw signal and to trigger acquisition of medical data from the patient in said medical data acquisition unit using said trigger signal.

14. A medical data acquisition system as claimed in claim 13 wherein said medical data acquisition device is a magnetic resonance data acquisition device comprising gradient coils that generate said interference noise.
